(19) United States
(12) Patent Application Publication
(10) Pub. No.: US 2023/0233146 A1
(43) Pub. Date: Jul. 27, 2023

(54) EDEMA DETECTION

(71) Applicant: TERUMO KABUSHIKI KAISHA, Tokyo (JP)

(72) Inventors: Joshua Brandon MYER, San Francisco, CA (US); Reza NAIMA, San Francisco, CA (US)

(21) Appl. No.: 18/009,685

(22) PCT Filed: Jun. 8, 2021
(86) PCT No.: PCT/IB2021/055004

§ 371 Date: Dec. 9, 2022

Related U.S. Application Data
(60) Provisional application No. 63/038,700, filed on Jun. 12, 2020.

(51) Int. Cl. A61B 5/00; A61B 5/0537

(52) U.S. Cl. CPC A61B 5/4878; A61B 5/0537; A61B 5/7221; A61B 5/746; A61B 5/6831; A61B 2560/0223; A61B 2562/043

(57) ABSTRACT

A method of controlling a wearable device including a signal generator, two stimulation electrodes, and two sensing electrodes to monitor a level of edema of a subject, includes generating, by the generator, a signal that causes a current to flow between the stimulation electrodes and measuring an impedance between the sensing electrodes disposed on a skin of the subject at an interval of time during a testing period, thereby providing impedance measurements, validating each impedance measurement against a model set of impedance measurements, eliminating a measurement from the impedance measurements if the measurement fails the validating, thereby providing a validated sub-set of impedance measurements, converting each of the validated sub-set of impedance measurements to an edema index, thereby providing edema indices, averaging the edema indices and generating an average edema index for the testing period, and generating an alert depending on the average edema index.

### CROSS REFERENCE TO RELATED APPLICATIONS

The present application claims priority to U.S. Provisional Patent Application No. 63/038,700 filed Jun. 12, 2020, which is herein incorporated by reference in its entirety.

### INCORPORATION BY REFERENCE

The present application also hereby incorporates herein by reference in its entirety, co-pending U.S. patent application Ser. No. 16/714,594 entitled “SYSTEMS AND METHODS FOR CALIBRATING DRY ELECTRODE BIOELECTRICAL IMPEDANCE SENSING,” filed Dec. 13, 2019 and published as U.S. Patent Application Publication No. 2020-0187823.

### TECHNICAL FIELD

Embodiments described herein related generally to a method of monitoring a level of edema of a subject and a wearable device.

### BACKGROUND

Being able to measure physiologic data in a non-invasive way that yields to high user compliance is critical in order to ensure continuous data is collected and acted upon for numerous health monitoring applications. While devices exist for non-invasive and, in some instances, passive monitoring of an individual over a period of time, the balance between comfort and the necessary secure disposition of sensors, such as electrodes, can create additional hurdles to realizing the benefits of these physiological monitoring techniques.

Depending on the measurement at issue, some physiological parameters are more difficult to measure than others and the data collection using non-intrusive monitoring can be challenging. The challenge is particularly great where non-intrusive monitoring is attempted for physiological parameters that are inherently difficult to measure and that may rely on devices that are subject to interference, the collection of erroneous data along with useful data, and the potential for introduction of an amount of erroneous data that overwhelms the collection of valuable data. Thus, the use of potentially valuable non-intrusive monitoring devices requires the development and design of devices that preferentially collect valuable data while enabling the dismissal of erroneous data, together with methods for data acquisition including ones that can retain qualified data while removing other data that may be inaccurate or erroneous.

### SUMMARY OF THE DISCLOSURE

One or more embodiments provide wearable devices and methods for using such devices to monitor the level of edema, e.g., level of hydration, fluid over-retention, or dehydration for an individual. The devices and methods include absolute or relative measurements of edema, measuring the change of edema over time by a variety of metrics, including measuring the change in the rate of change over time and assessing the impact of any of these metrics on a physiological condition. Such monitoring may be incorporated into other methods that are useful for acute monitoring situations such as dialysis, chemotherapy, exercise programs, post-surgical surveillance and any other physiological condition that may be accompanied by the need for absolute or relative change in levels or patterns of edema that may indicate an underlying physiological condition manifested by excessive fluids retention or dehydration reflected in a measurement of edema, which can leave an individual susceptible to an onset or progression of a number of adverse healthcare events, including infection, hypertension, kidney disease, cardiac disease, among others. Monitoring the level of edema may also be useful for long term scenarios for individuals with chronic heart failure, chronic kidney disease, and similar conditions where the subtle changes in absolute or relative measurements of edema may be the best indication of the progression of or remission from disease. The devices and methods described herein preferably are passive, e.g., not requiring active input from the individual or invasive monitoring that penetrates the skin or requires taking biological samples from a patient.

The wearable device is designed to comfortably contact the skin of the monitored individual to obtain an impedance measurement which may be converted to an edema index. In such devices, the lack of very close constriction of the wearable device around the skin of the patient, such as to avoid an uncomfortable restriction on blood flow or an uncomfortable restraint, can lead to inaccurate data or the excessive inclusion of inaccurate or erroneous data together with valuable data with an inability to separate what is clinically useful to treat a patient from that which confuses the diagnosis. However, the use of the devices as described herein and the methods for collecting and analyzing data require that the device and the data storage and processing features acquire a suitable number of data points with a high level of reliability, including particularly impedance values over a test period, and require the capacity to permit removal of inaccurate datapoints by a validation process. Accordingly, the development and processing of accurate data require the identification and removal of erroneous or invalid data that may properly be excluded on a medical or physiological basis from within a larger group of measurements used to calculate an edema index, that may represent a variety of physiological conditions, including but not limited to a dataset representing the hydration level of the individual.

Methods of monitoring and absolute or relative level of edema of a subject are provided together with, were necessary data processing and analytical steps including, but not limited to measuring an absolute or relative impedance value between at least two electrodes disposed at different points on a region of skin of an extremity of the subject both in absolute terms and over time and a change in the rate of change of an edema index calculated as described below. The set of measurements may be repeated at a selected interval of time during a period of testing, including separate and discrete periods of testing based on an identified calibration testing and protocol and may be comprised of providing a plurality of impedance measurements together with control and calibrated reference values. Each of the measurements are validated against a model set of impedance measurements collected in any of the testing, calibration, or control periods.

The methods include determining whether impedance measurements contained within any testing, control, or calibration protocol fail a validating process, that may exclude an individual data point or a set of data points such that erroneous or invalid data is identified and eliminated from the plurality of impedance measurements, and a validated sub-set of impedance measurements is provided. Each of the validated sets or subsets of impedance measurements are converted to an edema metric, including any of the calibrated edema indices described below that yield either of an individual or a plurality of edema indices derived from absolute or relative levels or patterns in the impedance measurements. The plurality of edema indices may be subject to mathematical processing including measurements of average, mode, median, threshold value, or mathematical or statistical measures to generate a particular edema index over the period of establishing a baseline, testing, or calibration. In some variations, there may be about 10%, 20%, 30%, 40% or more of the plurality of impedance measurements which may fail validation, and may be eliminated from a subset, or the final set of impedance values forming the validated set or sub-set of impedance measurements. In some variations, the sub-set of validated impedance measurements may include at least 40% of the plurality of impedance measurements measured during the period of testing.

Measuring the impedance may be repeated between once about every minute, about every 10 minutes, about every 20 minutes, about every 30 minutes, about every 60 minutes, or about once every 24 hours, or any period of time therebetween. Measuring the impedance may be performed for about 50 milliseconds, about 1 second, about 2 seconds, about 3 seconds, about 4 seconds, or any period of time therebetween. In some variations, the period of testing may be a period of about 1 hour to about 48 hours, or any value therebetween.

In some variations, the model set of impedance measurements may include a Cole-Cole model. Validating each of the plurality of impedance measurements may include fitting and evaluating individual, selected set or subset impedance measurements against the Cole-Cole model of impedance measurements. The Cole-Cole model provides several suitable edema index/metrics such as R₀, R_{inf}, f_{char}, and others. The quality of fit of the individual impedance measurements to the Cole-Cole model provides another measure of data quality: measurements which have markedly poorer quality of fit than a baseline expectation can be excluded from analysis. Examples of these measures include the total error term from the expected values given by the Cole-Cole model, the number of frequency points which lie beyond some threshold from the expected fit values, the overall shape of the data versus the Cole-Cole fit, the relationship between a given impedance measurement's Cole-Cole derived edema index and those from similar bioimpedance measurements (whether that similarity be determined by temporal proximity, or by proximity under some measure in a metric space defined by a combination of Cole-Cole features such as the edema metrics R₀, R_{inf}, and f_{char}; metadata about the sweep such as time of day; and features derived from the raw sweep itself such as the variance of the phase shift of the impedance signals).

The method may further include recording the average edema index for the period of testing. Measurements may be made over an extended duration of time, where the duration of time is at least a day and may extend to six months or more. In some variations, the specific statistical or mathematical calculation of an edema metric or index may be recorded for each period of testing over the extended duration of time.

In some variations, the method may include outputting or sending an alert when the selected edema metric exceeds a preselected value or range of values. In other variations, the method may further include outputting or sending an alert when the edema metric falls below a preselected or threshold value. The alert may be an electronic report to a patient, caregiver, or healthcare provider. In some variations, the alert may be an audible or visible report and may include data assembled by the wearable devices or data processed pursuant to the methods for using such devices.

In some variations, the different locations of the at least two electrodes may be at least a centimeter apart on the skin of the subject, or may be located anywhere on the individual's body, including configurations as distant as the electrodes located on opposing extremities such as one electrode on the left foot and the other on the right wrist.

In some variations, the method may further include securing one or more bands including at least two electrodes to the extremity of the subject, thereby disposing the at least two electrodes at the different points on the skin of the subject. In some variations, the extremity may be a wrist of the subject or a leg of the subject.

In any of the methods and apparatuses described herein the impedance measurements (which may be referred to as bioelectric impedance measurement) may be made by measuring electrical properties of biological tissue using one or more pairs of sensing electrodes, and determining an absolute, a relative, or a calibrated impedance measurement from an applied forward current in which current is applied between a pair of stimulation electrodes in a forward direction, as well as an applied shorted current in which the same current is applied simultaneously to both stimulation electrodes. The voltages at the sensing electrodes during both the forward operation (e.g., forward current) and the shorted operation (e.g., shorted current), as well as the current or voltage at a current sense resistor during the forward operation may provide a calibrated impedance measurement for the tissue. Sensing bioimpedance using this self-calibrated measurement, in which the ‘shorted’ current is used to calibrate the forward (and/or in some variations, reverse) current may provide highly accurate and reproducible results. The shorted current may be supplied before or after the forward (and/or reverse) current, and may be supplied immediately or shortly (e.g., within a few milliseconds, second or minutes) or the forward (and/or reverse) current. The same current may be supplied in the shorted configuration (e.g., same amplitude, frequency, duration, etc.) as in the forward and/or reverse current configuration(s). In some variations one or more properties (e.g., amplitude, frequency, duration, etc.) of the shorted current may be different from the forward and/or reverse current.

For example, methods of determining a bioelectrical impedance, which may be referred to as a “calibrated bioelectrical impedance” may include: supplying, in a forward mode, a first current between a source electrode and a sink electrode and storing voltages from a first sense electrode and a second sense electrode; supplying, in a shorted mode, a second current simultaneously to both the source electrode and the sink electrode and storing voltages from the first sense electrode and the second sense electrode; and outputting a calibrated bioelectric impedance measurement, wherein the bioelectric impedance measurement is based at least in part on the voltages of the sense electrodes in both the forward mode and the shorted mode. The first and second current may have the same amplitude, frequency, and/or duration or may be set to predetermined values that are recognized and reconciled and subsequent data processing steps. The first and second currents may be supplied within a predetermined time of each other (as close together as 100 microseconds, to as far apart as an hour). The first and second currents may be provided substantially immediately after one another. The method may include cycling between modes (e.g., between the forward mode and shorted mode or between forward, e.g., normal, mode, shorted mode and reverse modes).

Estimating the calibrated bioelectric impedance measurement may comprise determining the calibrated bioelectric impedance measurement based at least in part on: a voltage difference between the first and second sense electrodes in both the normal mode and the shorted mode; a ratio of voltages at the first sense electrode in the normal mode and the shorted mode; and a current across a current sense resistor in the normal mode.

### BRIEF DESCRIPTION OF THE DRAWINGS

FIG. 1 is a graphical representation of a wearable device according to some embodiments of the disclosure.

FIG. 2 is a block diagram of a wearable device according to some embodiments of the disclosure.

FIG. 3 is a block diagram illustrating a forward signal path through sense electrodes of a wearable device according to some embodiments of the disclosure.

FIG. 4 is a block diagram illustrating a reverse signal path through sense electrodes of a wearable device according to some embodiments of the disclosure.

FIG. 5 is a block diagram illustrating a shorted signal path through sense electrodes of a wearable device according to some embodiments of the disclosure.

FIG. 6 is a schematic representation of a five-element circuit model for bioimpedance measurements.

FIG. 7 is a graphical representation of a Cole-Cole plot for bioimpedance.

FIG. 8 is a flowchart of a monitoring process performed according to some embodiments of the disclosure.

FIG. 9 is a graphical representation of bioimpedance measurements over time.

FIG. 10A and FIG. 10B are graphical representations of the least squares fitting of bioimpedance measurements plotted against volume of extracted fluid.

FIG. 11 is a circuit diagram corresponding to a forward signal path and a reverse signal path of a wearable device according to some embodiments of the disclosure.

FIG. 12 is a circuit diagram corresponding to a shorted signal path of a wearable device according to some embodiments of the disclosure.

### DETAILED DESCRIPTION

Physiological monitoring may be a critical part of healthcare for individuals with chronic disorders such as, but not limited to, heart failure (which may also be called congestive heart failure (CHF)). In many disorders, monitoring hydration levels, such as edema and/or dehydration, can provide initial notice of a change in physical condition of the individual. An initial notice of such adverse change in hydration level can provide an opportunity for early intervention. The opportunity for early intervention can permit less drastic adjustment to medication, dialysis regimes, or personal care, instead of a later response to a more catastrophic change in condition of the individual. In addition to the obvious benefit to the individual, preventing serious deleterious change in condition also has cost benefits to a residential or out-patient care setting.

For example, heart failure patients may exhibit increased edema as disease spikes or progresses from a chronic to an acute condition where a pre-determined change in a metric of edema may indicate changes in pharmaceutical intervention, behavior changes, or even surgical intervention. The ability to intervene with more limited change in diuretic administration or introduction of other pharmaceutical agents without disturbing the effect of other medications that the individual may be using to control other disorders, can provide a more stable supportive regime. This can be particularly important for elderly patients who often are balancing care for a variety of disorders where changes in absolute or relative levels of an edema index may provide key information regarding a change in one or more underlying pathology.

In another example, monitoring hydration levels can be of importance in a post-surgical patient, when the patient is released to home or a rehabilitation center, having fewer healthcare professional interventions, to monitor that a post-surgical patient does not deteriorate into a dehydrated state, which can leave the post-surgical patient vulnerable to serious post-surgical secondary infections.

For these, and other conditions, physiological monitoring throughout a specified period of time, e.g., during a dialysis session for a patient having kidney failure, or for a less specific ongoing period of time, e.g., for a heart failure patient, may be advantageous. Use of monitoring devices requiring little or no input/assistance from the individual may be highly advantageous. Further, the device may need to be less tightly fitted to the individual in order to be comfortable for an individual in a residential care setting or for a post-surgical patient to obtain compliance. The device may also need to exclude certain features which generally provide higher efficiency, such as hydrogel pads for providing the skin-to-electrode contact often used in clinically used monitoring devices. As a result, the measurements obtained by the physiological monitoring device may be affected by movement of the individual, and may therefore not be accurate or useful and so the ability to separate erroneous or inaccurate data from valuable data under less than perfect physiological monitoring conditions is a valuable aspect of the present invention.

Therefore, methods of edema monitoring are needed that can test for, and eliminate, data points or datasets that are erroneous, inaccurate or that otherwise do not provide usable monitoring data. While devices worn for extended periods can provide such erroneous data, as described above, the continued operation of the device for an extended period of data collection can offer the offsetting advantage of acquiring a multiplicity of data, which can be subsequently tested to determine which of the data should be included in obtaining a measurement of edema and/or dehydration and which of the data should be rejected as inaccurate or erroneous.

Bioelectrical impedance analysis measures the bioimpedance of the body tissues produced within a body part of an individual when an alternating current tends to flow through it. Bioimpedance is a function of tissue properties as well as the applied current signal frequency. The human body contains several different components that contribute to these measurements including minerals (such as those found in bone and electrolytes), muscle and lean tissue mass; and body water, which is divisible into intracellular and extracellular water. Furthermore, discrete intracellular structures may also contribute to an edema index and may be separable from intracellular and extracellular measurements or may be processed using separate statistical metrics as part of the analytical methodology described herein.

As cell membranes are capacitive in nature, the capacitive reactance produced by the electric current allows the current pass through, individually or collectively, such structures depending on the signal frequency and hence the current paths. Low frequency current passes through extracellular fluids as the cell membrane reactance does not allow the low frequency current to pass through such structures whereas the high frequency current penetrates the cell membranes and passes through both the extracellular fluids and the cells (membranes and intracellular fluids). Thus, by applying the alternating current at a particular frequency, bioimpedance measurement can assess the amount of extracellular water (ECW), intracellular water (ICW), and total body water (TBW=ECW+ICW). Measurement of edema and/or dehydration can be thus obtained.

Methods are provided here for monitoring a level of edema and/or dehydration, more generally monitoring the hydration level of an individual, using a wearable device. The level of hydration may be obtained from measurements of impedance across body portions of the individual.

The wearable device may have any number of driving, sensing, or combined driving/sensing electrodes such that two driving points and two sensing points may be chosen. A wearable device having a two-electrode configuration provides current signal injection and voltage measurement at the same electrodes. The impedance measured by a two-electrode device therefore, includes a voltage drop due to contact impedance. In a wearable device having a four-electrode configuration, two separate electrode pairs are used for current injection and voltage measurements, and a constant amplitude current signal may be input through the two outer electrodes (e.g., current electrodes or the driving electrodes) and frequency dependent voltage signals may be measured across two points through the two inner electrodes (e.g., voltage electrodes or sensing electrodes). In any case, the wearable device may be configured to measure impedance between two electrodes located at different locations upon an extremity of the individual. The extremity of the individual may be an arm or a portion of the arm, such as a wrist, or may be a leg or a portion of a leg, such as an ankle. In some variations, the wearable device is configured to be secured to the wrist of an individual, such that the two electrodes which sense voltage (e.g., convertible to an impedance measurement) are disposed at two different points upon the skin of the individual. The two points may be separated from each other by at least 10 mm, or as far apart as allowed by the physiology of the subject wearing it. The sensor electrodes are localized and read the difference for the region between the two sensing electrodes. The voltage detected can be converted to impedance (Z) which represents a measure of hydration in the body part traversed by the input current. In the methods provided here, impedance may be correlated to an edema index for monitoring the individual.

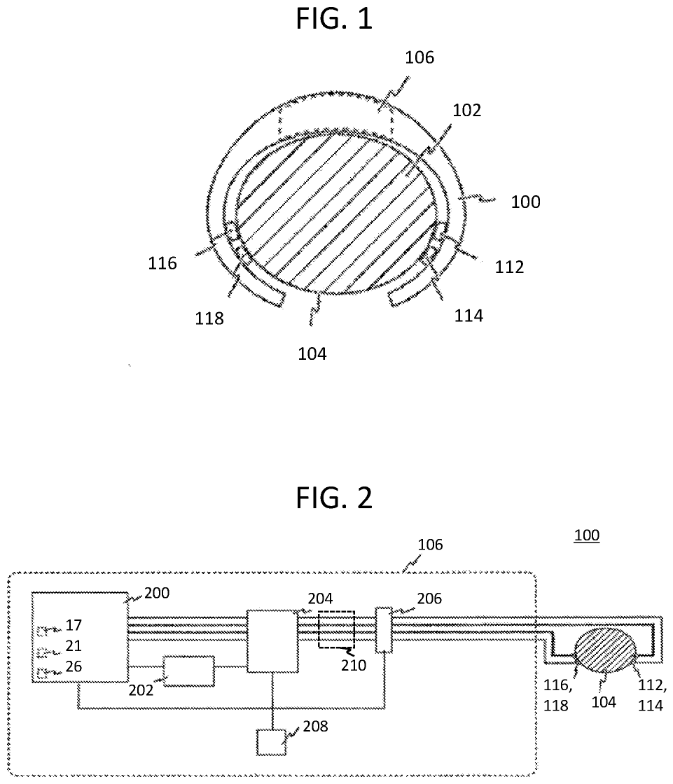

FIG. 1 shows one non-limiting example of a suitable wearable device 100. The bioelectric measurement wearable device 100 of FIG. 1 is shown engaging the wrist 102 of an individual, where the wearable device 100 contacts the skin 104 of the individual. The wearable device 100 includes internal electronics 106 which are connected to electrodes 112, 114, 116, and 118, which contact the skin 104. The first electrode 112 and the third electrode 116 are stimulation electrodes. The second electrode 114 and the fourth electrode 118 are sensing electrodes. The electrodes are all dry contact electrodes and require no skin preparation, gel or other material to optimize the skin-electrode impedance.

The wearable device 100 may be electronically connected to other devices, such as processors, medical records or databases, where the data may be processed locally within the device or within nearby or remote processors. The wearable device 100 may be further configured to provide a visible signal if battery power is low. The wearable device 100 may be further configured to provide a visible or audible alert if measurements, once validated as described below, exceed a preselected threshold value and/or fall below a preselected threshold value. To achieve these functions, the wearable device 100 may have one or more lamps such as LEDs, a display such as a liquid crystal display (LCD), and/or a speaker (not shown in FIG. 1).

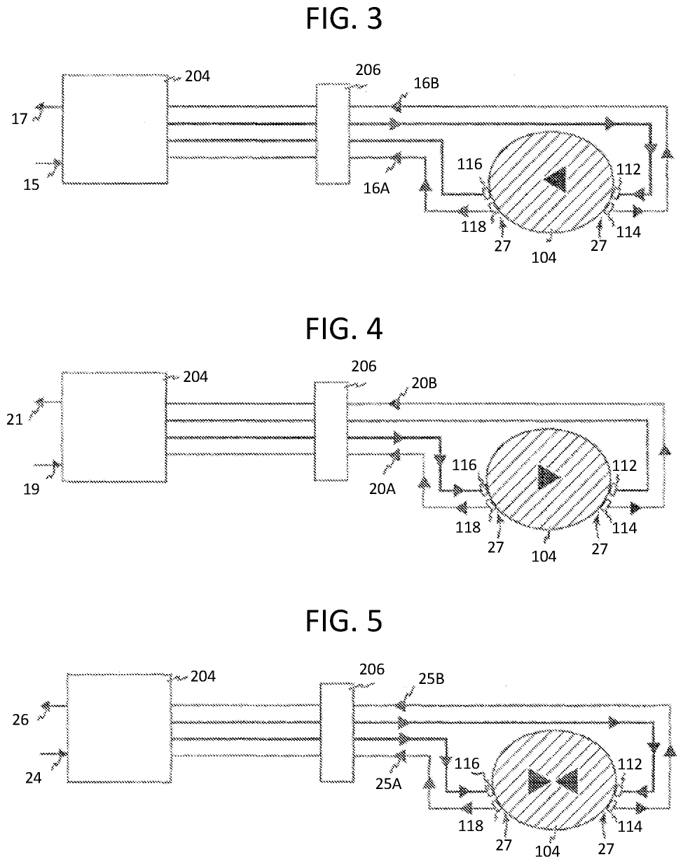

FIG. 2 is a block diagram of the wearable device 100. As shown in FIG. 2, the internal electronics 106 of the wearable device 100 include: a controller 200, a signal generator 202 for generating test signals, a signal processer 204 for processing signals transmitted to or received via the electrodes 112, 114, 116, and 118, an optional multiplexer 206 for multiplexing and routing signals, a power source 208 for supplying power such as a battery, and a current sense resistor 210 for sensing a current. For example, the current sense resistor 210 is electrically connected to any of the lines between the signal processor 204 and the multiplexer 206 to sense a current flowing therebetween. The controller 200 may include one or more processors and volatile and non-volatile memories. The controller 200 may further include an interface circuit configured to communicate with an external device such as a host computer to output an alert and related data via a wired or wireless network. In some variations, any of these components may be combined or integrated together. Characterization of the skin-electrode interface may be accomplished by routing a test signal generated by the signal generator 202 through the optional multiplexer 206 or other control and/or switching circuitry and through the stimulation electrodes 112 and 116 in a forward configuration or a shorted configuration (and in some variations, in a reverse configuration) as described and illustrated below in FIGS. 3-6.

For example, FIG. 3 illustrates one example of the operation of the wearable device 100 shown schematically in FIG. 2 in a forward configuration. In the forward configuration, the controller 200 may be configured to operate the wearable device 100 so that a signal 15 generated by the signal generator 202 is passed and/or processed by the signal processor 204 and routed by the multiplexer 206 in a forward direction between the source electrode 112 and the sink electrode 116. As the current flows between the source and sink electrodes 112 and 116, the controller 200 may detect signals from the sense electrodes 114 and 118. In this example, data signals 16A and 16B may be processed and interpreted by the signal processor 204 and the controller 200. These data signals may correspond to voltage(s) at the sense electrodes 114 and 118. Concurrently, a signal (e.g., voltage and/or current) from the current sense resistor 210 (not shown in FIGS. 3-5) may be recorded during the application of the forward signal, and the resulting forward characteristic data 17 may be stored in a memory (not shown) of the controller 200 as shown in FIG. 2.

Following operation of the wearable device 100 in the forward configuration for one or more set of samples (e.g., recording at one or more frequencies, etc.), the wearable device 100 may be automatically (e.g., by action of the controller 200) switched to operate in the shorted configuration. Alternatively or additionally, the wearable device 100 may be configured to switch to operate in the reverse configuration, in which the source and sink electrodes 112, 114, 116, and 118 may be reversed (e.g., the source may operate as the sink and the sink as the source) as illustrated in FIG. 4.

FIG. 4 illustrates one example of the operation of the wearable device 100 shown schematically in FIG. 2 in a reverse configuration. In FIG. 4, a signal 19 generated by the signal generator 202, which may be the same or different from the signal 15 applied in the forward configuration, may be processed by the signal processor 204 and routed by the multiplexer 206 in a reverse direction, between the sink electrode 116 and the source electrode 112. The sense electrodes 114 and 118 may be used to record data signals 20A and 20B (e.g., voltages) arising from the reverse current, and these sense data signals may be processed and interpreted by signal processor 204 and the controller 200, along with the sensed current and/or voltage from the current sense resistor 210, and the resulting reverse or second characteristic data 21 may be stored by the controller 200 as shown in FIG. 2.

As mentioned above, immediately following one or more operations of the wearable device 100 in the forward and/or reverse configuration or modes, the wearable device 100 may be automatically (e.g., by action of the controller 200) switched to operate in the shorted configuration, in which current is sent to both the source and sink electrodes 112 and 116 simultaneously. In the shorted configuration or shorted mode, the same current may be supplied to both the source and sink electrodes 112 and 116. The supplied current may be the same or approximately the same as supplied during the forward and/or reverse configuration. In some variations the current may be different, e.g., the current supplied to both electrodes when operating in the shorted configuration may be less than during operation in the forward and/or reverse configuration.

FIG. 5 illustrates one example of the operation of the wearable device 100 described above in the shorted configuration/mode. In FIG. 5, parallel signals 24 (e.g., current) generated by the signal generator 202 may be processed by the signal processor 204 and routed by the multiplexer 206 in a parallel or shorted direction so that the same signal (e.g., current) is supplied to both the source electrode 112 and the sink electrode 116. The signal sensed by the sense electrodes 114 and 118 arising from the supplied signal may be received as data signals 25A and 25B and processed and/or interpreted by the signal processor 204 and the controller 200. The received signals (e.g., voltage at the sense electrodes 114, 118) during the shorted operation may correspond to shorted characteristic data 26, and may be stored by the controller 200 as shown in FIG. 2.

In some variations, the controller 200 uses the forward data 17 and shorted data 26 (and/or in some embodiments, the reverse data 21 and shorted data 26) to characterize the interface 27 between the electrodes 112, 114, 116, and 118 and the skin and to determine an accurate estimation of the bioelectric signals (e.g., bioelectric impedance) of the tissue (i.e., the skin in contact with the electrodes).

Using the wearable device 100 described above, impedance measurements may be taken frequently, thereby supplying a plurality of impedance measurements, which may be as low as 10 measurements, up to many more (e.g., several thousands) over a selected period of testing. The measurements may be made at a selected interval of time during the period of testing. Impedance measurement may be repeated as frequently as about every minute to once per 48 hours. An impedance measurement may collect data for between 500 μs through to 450 s.

The measurements may be collected over a period of testing, which may be for about 1 hour, about 2 hours, about 4 hours, about 6 hours, about 8 hours, about 12 hours, about 16 hours, about 18 hours, about 24 hours, or any value therebetween. In some variations, the period of testing may be about 1 hour to about 24 hours. The measurements taken over a period of testing may be grouped together to obtain an averaged value of the measurement or any other statistical grouping deemed to purvey meaningful information to the caregiver, e.g., a daily averaged value of the measurements. The grouped value of the measurement, which may be represented as an edema index or hydration value, may be included in records for the individual and retained over a duration of time for which measurements are made.

The measurements, while grouped from a shorter period of testing, such as over a daily basis, as in one non-limiting example, may be continued over a longer duration of time to monitor the individual. The duration of time for which monitoring is provided may be a short duration, such as one day, two days, or a few days, such as when monitoring an individual undergoing kidney dialysis. In other variations, such as when monitoring an individual with heart failure, the duration of time for which monitoring is provided may be about a week, about one month, about two months, about 3 months, about 6 months, a year or more. In some variations, monitoring may be provided from about 1 month to about 6 months, or longer. The averaged (or grouped) value from each period of time of testing may be recorded throughout the duration of the monitoring. In some variations, the value that is recorded may be an averaged edema index, rather than the impedance value.

In some variations, an alert may be output or sent from the wearable device 100, an external device to which the wearable device is connected, or an external device storing the database to which the grouped or averaged measurements are sent, when the averaged/grouped measurement exceeds a preselected threshold or falls below a preselected threshold. The alert may inform the patient, healthcare providers or caregivers that intervention may be necessary or more involved monitoring may be needed for the individual. In some variations, the alert may further include a visible or audible alert issued from the wearable device 100, the external device to which the wearable device 100 is connected, or the external device storing the database to which the grouped or averaged measurements are sent. Accordingly, the device and methods of data analysis may be integrated with companion devices carried by the patient, such as cell phones, computers, and other mobile monitoring devices, as well as institutional networks employed by hospitals for localized or decentralized monitoring of patients having a chronicle or critical care condition.

As noted above, individual impedance measurements, may be or may include erroneous factors, since the wearable device 100 is not uncomfortably tightly secured against the body portion of the individual and the electrodes do not have hydrogel or other skin preparations to assist in the electrical measurements. Additionally, the individual is not required to maintain a restrained or immobilized position. Under any of these conditions, the wearable device 100 may slip or move and yield an erroneous measurement. Thus, the wearable device 100 can validate any or all of the impedance measurements.

The impedance measurements may be made at a single frequency or may be made at multiple frequencies and may be from about 1 kHz to about 1 MHz, or any single or multiple frequencies therebetween.

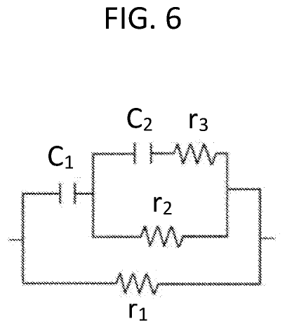

Impedance measurements may be validated against a model for bioimpedance, which may be selected to be a five element circuit model, as shown in FIG. 6. In this model, r₁ represents extracellular fluid, and the other branch represents the intracellular component of aqueous fluid containing structures. C₁ represents cellular membranes and r₂ intracellular (cytoplasmic) fluid. C₂ represents intracellular membranes and r₃ the corresponding fluids within the intracellular structures (e.g., nucleus, lysosome, etc.) bounded by the intracellular membranes. In some variations of these techniques, the C₂-r₃ branch of this circuit may be disregarded and modeled via adjustments to the C₁ and r₂ values.

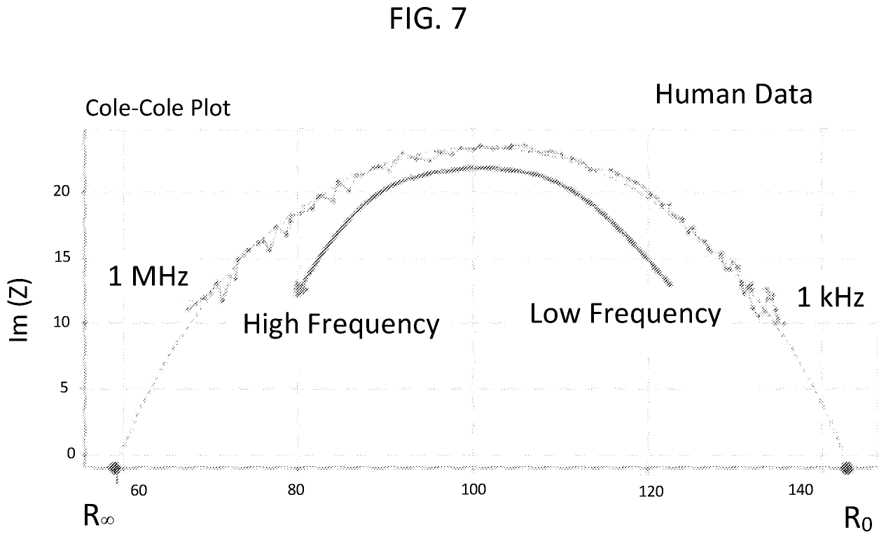

Each of the data points, or any selected sub-set of them may be fitted against the Cole-Cole model as shown in FIG. 7, using the following relationship:

$$Z ( ω ) = {R}_{∞} + \frac{{R}_{0} - {R}_{∞}}{1 + {( j ω r )}^{α}} \qquad [ Math . 1 ]$$

$$Z ( ω ) = ( {R}_{∞} + \frac{{R}_{0} - {R}_{∞}}{1 + {( j ω r )}^{α}} ) exp ( - j ω {T}_{d} ) \qquad [ Math . 2 ]$$

However, a data point, or a set of data points, that does not produce a good fit against the Cole-Cole plot, may be eliminated from the plurality of impedance measurements, thereby providing a validated sub-set of impedance measurements. In some variations, there may be about 5%, about 10%, about 15%, about 20%, about 30%, about 40%, or more of the impedance measurements taken during a period of testing which may fail validation, e.g., fail fitting to the Cole-Cole plot.

Each of the validated impedance measurements may then be used to compute an individual edema index using a combinations of the values derived from equations. One example of an edema index is to use just the Ro term. Another example is the following relationship:

$$\frac{{R}_{0}^{- 1}}{{R}_{∞}^{- 1} - {R}_{0}^{- 1}} \qquad [ Math . 3 ]$$

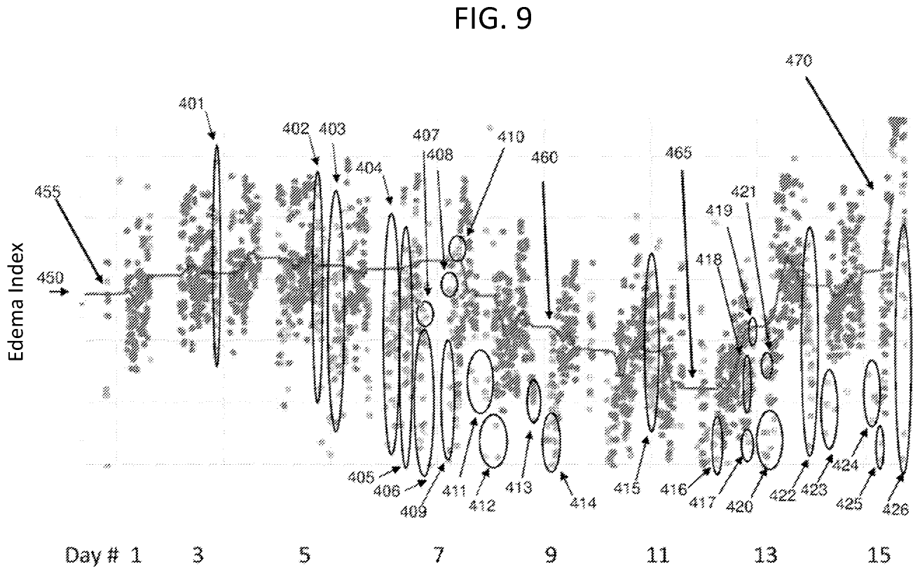

The wearable device 100 thereby provides a plurality of edema indices per period of testing, which may be averaged in any suitable manner to provide an average edema index for the period of testing. An advantage of this method is that large numbers of impedance measurements may be taken, without any input from the monitored individual, permitting the exclusion of the proportion of impedance measurements which do not fit to the Cole-Cole plot. In some variations, the validated measurements may be more than about 25%, about 30%, about 40%, about 50%, about 60%, about 70%, about 80% or more of the impedance measurements taken during the period of testing. The resultant edema metric, including but not limited to an averaged edema index for the period of testing correlates with the state of hydration of the individual, even if there are many data points which could not be included due to motion of the wearable device 100 upon the skin of the individual, as shown in FIG. 9 and discussed further below.

The period of testing may be, for example, a 24 hour period, and the duration of monitoring may be for several days or for many days, and the average edema index may be used to track the edema level (or hydration level) for an individual monitored by the wearable device 100.

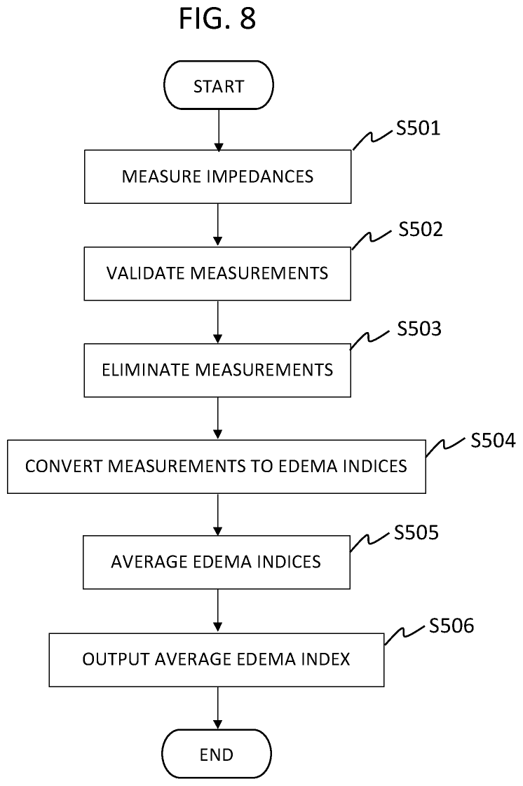

FIG. 8 is a flowchart showing the steps of the monitoring method performed by the wearable device 100. Initially, the controller 200 controls the signal generator 202 to generate a testing signal that causes a particular current to flow between the stimulation electrodes 112 and 116 disposed on the skin of an extremity of the subject, e.g., an arm, with various frequencies and measures impedances between two sensing electrodes 114 and 118. The measured impedances may be stored in a memory of the controller 200 (not shown).

At S502, the controller 200 validates the impedances measured at S501 by determining whether each impedance fits against the Cole-Cole model. For example, the controller 200 determines that one or more of the measured impedances that fall within a particular range with respect to the Cole-Cole plot shown in FIG. 7 fit with the Cole-Cole model. At S503, the controller 200 eliminates the measured impedances that have not been validated at S502 from the subsequent analysis. At S504, the controller 200 converts the validated impedances to edema indices. For example, the conversion can be made based on Math. 3 described above. Subsequently, the controller 200 calculates the average of the edema indices at S505, and outputs the averaged edema index at S506, by storing the index in the memory, for example. The controller 200 may transmit the calculated edema index to an external device via a network interface (not shown).

All of the steps illustrated in FIG. 8 can be performed by the wearable device 100. Alternatively, one or more of S503-S506 may be performed by an external device connected to the wearable device 100. In such a case, the controller 200 transmits the measured impedances to the external device via an interface circuit. The steps illustrated in FIG. 8 may repeatedly performed during a selected testing period.

### EXAMPLE

Experiment 1. Monitoring an individual over an extended period. A subject was fitted with the wearable device 100 and was monitored passively over a 15 day period, collecting impedance measurements over 10 to 20 minutes during selected periods of time each day as shown in FIG. 9. The data was collected during waking periods for the individual, and were not distributed evenly throughout each 24 hours period. Data was validated as described above, against the Cole-Cole model. Data that did not validate are shown substantially within regions 401-426. Only validated data points were used in conversion to edema indices, with subsequent averaging to produces an averaged edema index for each time period (i.e., daily). The daily average edema index is indicated as line 450 from day #1 to day #15.

As can be seen in FIG. 9, the average edema index value, shown in line 450 decreases from day #1 (i.e., the average edema value at point 455), as the individual suffered from an influenza. The averaged edema index decreased with increasing dehydration due to influenza, at point 460 on day #9, to a low at point 465 on day #12. As the individual recovered, the average edema index also rebounded, as shown at point 470 at day #15.

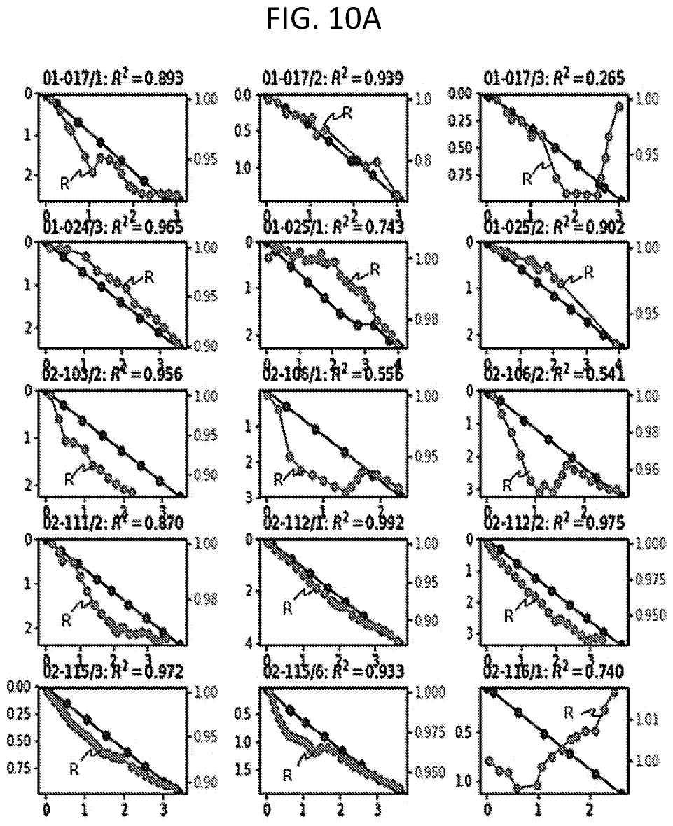

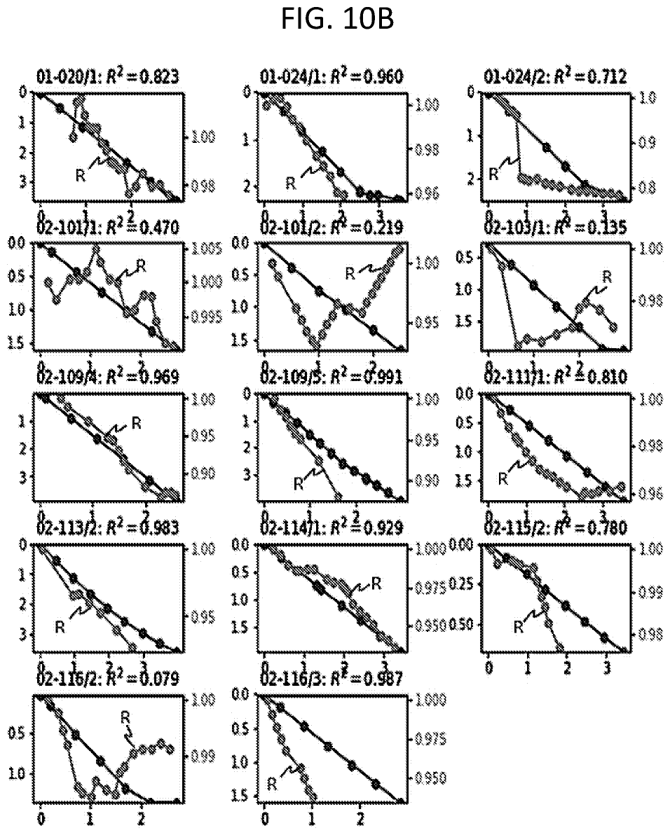

Experiment 2. Edema measurements during dialysis. A group of individuals undergoing dialysis were fitted with the wearable device 100 and monitored over the period of dialysis, conducted over a three hour period. Measurements were made every ten to twenty minutes, and measurements were fitted against the Cole-Cole model as described herein. Each panel in FIG. 10A and FIG. 10B shows an individual dialysis session for one individual. The panel shows the course of time along the X axis, and the volume of water (liter, L) extracted from time t=0 to 3 hours, is shown decreasing from a value of 0.00 to a final extracted volume (i.e., the intersection of the left hand Y1 axis with X axis). The Y1 axis shows the inverted amount of ultrafiltration recorded during a dialysis session. The hydration level of the individual, as measured by the methods described herein, is shown at the right hand Y2 axis, decreasing from a value of 1.00 to a final value at t=3 hours. The right hand Y2 axis shows a value of R₀ immediately after the start of the dialysis session divided by each value of R₀ during the dialysis session (i.e., normalized resistance). For illustration, only the graph of Y2 axis is labelled “R” in each panel. The least squares value R², representing the goodness of fit ranges from a high of 0.973 to a low of 0.135.

As shown in the set of graphical panels in FIG. 10A and FIG. 10B, the correspondence from individual to individual, and from dialysis session to a succeeding dialysis session, did not achieve complete correspondence, but over the entire set of individual measurement sessions, an overall value of 0.752 for R² was obtained, indicating a substantial correspondence for the entire group.

### Calibration of Bioelectric Impedance

As mentioned above, the shorted configuration of the wearable device 100 as shown in FIG. 5 may be used to calibrate the bioelectric impedance. Such calibration is made upon or after the impedance measurement shown in FIG. 8, for example.

The impedance mismatch between the subject's skin 104 and the sensing electrodes 114 and 118 can be determined by the controller 200 of the wearable device 100 during calibration and used to adjust the interpretation of bioelectric signals from the electrodes 114 and 118. The wearable device 100 performs a set of calibration measurements. For example, the calibration measurements may include the differential voltage between the sense electrodes 114 and 118, the total current through the electrodes 114, 118 (e.g., the current through the current sense resistor 210), and the voltage at the input of one of the sense electrodes 114, 118 during the forward (or reverse) and shorted configurations. Any suitable set of measurements may be used to calibrate the impedance of the electrode/skin interface 27.

For example, as described above, a first set of measurements may be made with the current flown in either the forward or the reverse direction to provide the forward data 17, or the reverse data 21. The shorted data 26 may be collected as discussed above (e.g., immediately after, before or intermittently with collecting the data from the forward and/or reverse configuration), and the first set of data, e.g., the forward data 17, and the shorted data 26 may be combined to calculate a first impedance of subject's tissue that is calibrated by the use of the shorted data.

In some variations, the measurement and calculation process may be repeated using the previously unused current direction (e.g., the reverse data 21) and the corresponding shorted data 26. The reverse data 21 and the shorted data 26 may be combined to calculate a second impedance of subject's tissue. The first impedance data may then be combined with the second impedance data, e.g., by averaging the two together, by weighting the forward with the reverse, etc., which may improve the accuracy of the resulting bioelectric impedance measurements.

Specifically, the bioelectric impedance may be calibrated by the differential voltage at the sense electrodes 114 and 118 and a ratio of the voltage at the input to one of the sense electrodes 114 and 118 during both the forward and shorted configurations.

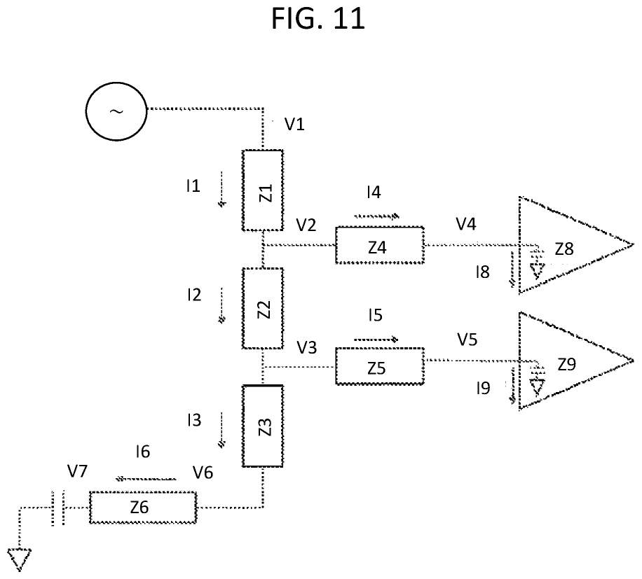

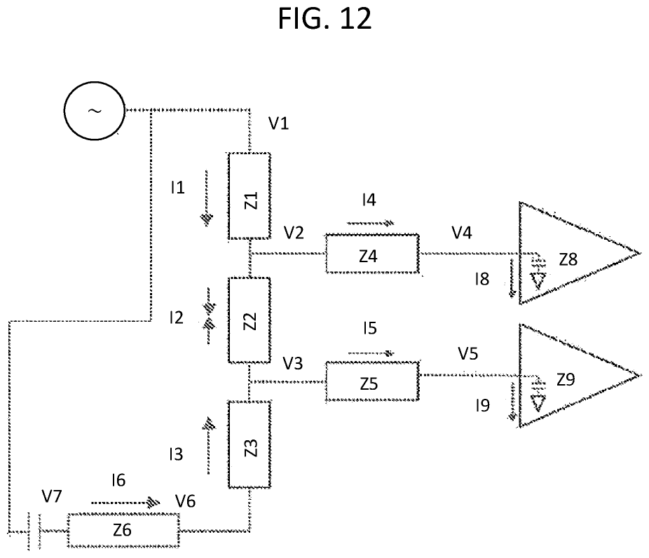

For example, FIGS. 11 and 12 show schematic diagrams of the operation of the wearable device 100 shown in FIGS. 2-5 during a normal, forward configuration and during a shorted configuration, respectively. In FIGS. 11 and 12, the current source/sink electrodes 112 and 116 have impedances Z1 and Z3, respectively. The voltage sense electrodes 114 and 118 have impedances Z4 and Z5, respectively. The subject's tissue has an impedance Z2, and the current sense resistor 210 has an impedance Z6. Z8 and Z9 refer to the input impedances to buffer amplifiers. As mentioned above, FIG. 11 illustrates a system in the forward configuration or mode and FIG. 12 illustrates the system operating in the shorted configuration or mode, with the current simultaneously applied to both the source electrode 112 and the sink electrode 116.

As described above, the wearable device 100 has the source electrode 112 and the sink electrode 116, and at least two sense electrodes 114 and 118. The wearable device 100 also has the capability of switching between the forward or normal configuration and the shorted configuration, and in some variations also the reverse configuration. Thus, the wearable device 100 may be configured to direct current in a forward direction (and/or a reverse direction) as well as directing current into both source and sink electrodes 112 and 116 simultaneously, enabling the measurement of leakage currents I8 and I9, shown in FIGS. 11 and 12, through the sense electrodes 114 and 118.

Thus, the wearable device 100 may be configured to measure the differential voltage multiplied by the gain of the amplifier at the sense electrodes: (G(V₄−V₅))=β. In the normal (i.e., forward) configuration, the differential voltage at the sense electrode multiplied by the gain may be indicated by the subscript “N.” In the shorted configuration, the differential voltage at the sense electrode multiplied by the gain may be indicated by the subscript “B.” Thus:

β_{N}=G(V_{4,N}−V_{5,N}) [Math. 4]

β_{B}=G(V_{4,B}−V_{5,B}) [Math. 5]

The differential voltage across the current sense resistor 210 is: (G(V₆−V₇))=α. Thus, for the forward configurations:

α_{N}=G(V6−V7)=I_{6,N}Z₆G [Math. 6]

The various gains indicated above may be set to be the same gain (e.g., the gains for the amplifiers used) or they may be different gains; for convenience, these gains are shown herein as being the same gain, however it should be understood that they may be different.

The voltage at the input of one of the sense electrodes 114 and 118, e.g., V₄ is γ. For the forward and shorted configurations, respectively:

γ_{N}=V_{4,N} [Math. 7]

γ_{B}=V_{4,B} [Math. 8]

The following set of equations describes the current flowing in the forward direction:

V₄−V₅=(V₂−Z₄I₄)−(V₃−Z₅I₅) [Math. 9]

V₄−V₅=(V₂−V₃)+(Z₅I₅−Z₄I₄) [Math. 10]

Where V₂−V₃ represents the measurement to be made and (Z₅I₅−Z₄I₄) represents the error term. Using the following equalities:

V₂−V₃=Z₂I₂ [Math. 11]

I₂=I₆+I₉ [Math. 12]

it is possible to derive the relationship:

V₄−V₅=Z₂(I₆+I₉)+Z₅I₅−Z₄I₄ [Math. 13]

Because of the relationships: I₅=I₉ and I₄=I₈,

V₄−V₅=Z₂(I₆+I₉)+Z₅I₉−Z₄I₈ [Math. 14]

As mentioned above, the normal (e.g., forward/reverse) current operation may be indicated by the subscript of N in the measurement terms. Under this condition, I₆>>I₉, and the relationship simplifies to:

V_{4,N}−V_{5,N}=Z₂I_{6,N}+Z₅I_{9,N}−Z₄I_{8,N} [Math. 15]

For the shorted mode where current is supplied to both the source and sink electrodes 112 and 116 simultaneously, I₆+I₉=I₂ and approximately equal to I₈, which is also approximately equal to I₉. However, because of the relationships Z₂<<Z₄ and Z₂<<Z₅, Z₂I₂ can be set to zero. This assumption simplifies the relationship to:

V_{4,B}−V_{5,B}=Z₅I_{9,B}−Z₄I_{8,B} [Math. 16]

Substituting in:

$${I}_{9 , B} = \frac{{V}_{5 , B}}{{Z}_{9}} and {I}_{8 , B} = \frac{{V}_{4 , B}}{{Z}_{8}} \qquad [ Math . 17 ]$$

results in:

$${V}_{4 , B} - {V}_{5 , B} = {Z}_{5} ( \frac{{V}_{5 , B}}{{Z}_{9}} ) - {Z}_{4} ( \frac{{V}_{4 , B}}{{Z}_{8}} ) \qquad [ Math . 18 ]$$

The ratio of voltages (e.g., V₄/V₅) is fairly consistent, independent of the mode of operation. This has been validated empirically. In some variations, an additional measurement at V₅ may be used to obviate the need for this approximation. Using the relationship:

$$\frac{{V}_{4 , N}}{{V}_{5 , N}} \approx \frac{{V}_{4 , B}}{{V}_{5 , B}} \qquad [ Math . 19 ]$$

and rearranging to

$$\frac{{V}_{4 , N}}{{V}_{4 , B}} \approx \frac{{V}_{5 , N}}{{V}_{5 , B}} \qquad [ Math . 20 ]$$

and multiplying both sides of Math. 18, result in:

$$( {V}_{4 , B} - {V}_{5 , B} ) ( \frac{{V}_{4 , N}}{{V}_{4 , B}} ) = {Z}_{5} ( \frac{{V}_{5 , B}}{{Z}_{9}} ) ( \frac{{V}_{5 , N}}{{V}_{5 , B}} ) - {Z}_{4} ( \frac{{V}_{4 , B}}{{Z}_{8}} ) ( \frac{{V}_{4 , N}}{{V}_{4 , B}} ) \qquad [ Math . 21 ]$$

This can be simplified to:

$$( {V}_{4 , B} - {V}_{5 , B} ) ( \frac{{V}_{4 , N}}{{V}_{4 , B}} ) = {Z}_{5} ( \frac{{V}_{5 , N}}{{Z}_{9}} ) - {Z}_{4} ( \frac{{V}_{4 , N}}{{Z}_{8}} ) \qquad [ Math . 22 ]$$

In view of:

$$\frac{{V}_{5 , N}}{{Z}_{9}} = {I}_{9 , N} \qquad [ Math . 23 ]$$

$$and$$

$$\frac{{V}_{4 , N}}{{Z}_{8}} = {I}_{8 , N} , \qquad [ Math . 24 ]$$

$$( {V}_{4 , B} - {V}_{5 , B} ) ( \frac{{V}_{4 , N}}{{V}_{4 , B}} ) = {Z}_{5} {I}_{9 , N} - {Z}_{4} {I}_{8 , N} \qquad [ Math . 25 ]$$

Subtracting Math. 25 from Math. 15 produces:

$$( {V}_{4 , N} - {V}_{5 , N} ) - ( {V}_{4 , B} - {V}_{5 , B} ) ( \frac{{V}_{4 , N}}{{V}_{4 , B}} ) = {Z}_{2} {I}_{6 , N} + {Z}_{5} {I}_{9 , N} - {Z}_{4} {I}_{8 , N} - {Z}_{5} {I}_{9 , N} + {Z}_{4} {I}_{8 , N} \qquad [ Math . 26 ]$$

Canceling results in:

$$( {V}_{4 , N} - {V}_{5 , N} ) - ( {V}_{4 , B} - {V}_{5 , B} ) ( \frac{{V}_{4 , N}}{{V}_{4 , B}} ) = {Z}_{2} {I}_{6 , N} \qquad [ Math . 27 ]$$

Finally solving for the issue impedance (Z₂), results in:

$${Z}_{2} = \frac{( {V}_{4 , N} - {V}_{5 , N} ) - ( {V}_{4 , B} - {V}_{5 , B} ) ( \frac{{V}_{4 , N}}{{V}_{4 , B}} )}{{I}_{6 , N}} \qquad [ Math . 28 ]$$

Thus, measuring all the terms in the above equation will provide a calibrated impedance of the tissue of the subject.

The equalities described above may be used in Math. 28 to result in:

Z₂=(β_{N}−β_{B}*(γ_{N}/β_{B}))/(α_{N}/Z₆) [Math. 29]

The preceding analysis assumes that under normal operations, I₆>>I₉ and thus, the Z₂I₉ term in Math. 14 may be set to zero. In the situation where the current is supplied to both current paths (e.g., the shorted configuration), I₆+I₉=I₂ and is approximately the same as I₈ and I₉, and Z₂<<Z₄, and Z₂<<Z₅, allowing Z₂I₂=Z₂(I₆+I₉) in Math. 11 be set to zero. Finally, the ratio of the voltages V₂ to V₅ may be the same in the normal and shorted modes, thus the ratio of V_{4,N}/V_{4,B} is approximately equal to V_{5,N}/V_{5,B}.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A method of controlling a wearable device including a signal generator, at least two stimulation electrodes, and at least two sensing electrodes to monitor a level of edema of a subject, the method comprising:
generating, by the signal generator, a first signal that causes a current to flow between the at least two stimulation electrodes and measuring an impedance between the at least two sensing electrodes disposed on a skin of the subject at a selected interval of time during a period of testing, thereby providing a plurality of impedance measurements;
validating each of the plurality of impedance measurements against a model set of impedance measurements;
eliminating an impedance measurement from the plurality of impedance measurements if the impedance measurement fails the validating, thereby providing a validated sub-set of impedance measurements;
converting each of the validated sub-set of impedance measurements to an edema index, thereby providing a plurality of edema indices;
averaging the plurality of edema indices and generating an average edema index for the period of testing; and
generating an alert depending on the average edema index.

2. The method of claim 1, wherein
the at least two stimulation electrodes include a source electrode and a sink electrode, and
measuring the impedance includes measuring a calibrated bioelectric impedance by supplying a first current between the source electrode and the sink electrode in a first direction, supplying a second current simultaneously to the source electrode and sink electrode in the first direction and a second direction opposite to the first direction, and calculating the calibrated bioelectric impedance based at least in part on voltages between the at least two sense electrodes when the first and second currents are supplied.

3. The method of claim 2, wherein calculating the calibrated bioelectric impedance includes determining the calibrated bioelectric impedance based at least in part on:
a voltage difference between the at least two sense electrodes when each of the first and second currents is supplied;
a ratio of voltages at a first sense electrode when the first and second currents are supplied; and
a current that flows across a current sense resistor when the first current is supplied.

4. The method of claim 1, wherein measuring the impedance is repeated between once every ten minutes to once every twenty minutes.

5. The method of claim 1, to wherein measuring the impedance is performed for 1 second to 4 seconds.

6. The method of claim 1, wherein the period of testing is a period of 1 hour to 24 hours.

7. The method of claim 1, wherein the model set of impedance measurements includes a Cole-Cole plot.

8. The method of claim 7, wherein validating each of the plurality of impedance measurements includes fitting each impedance measurement against the Cole-Cole plot of impedance measurements.

9. The method of claim 1, further comprising:
recording the average edema index for the period of testing.

10. The method of claim 1, further comprising:
extending the period of testing to a predetermined duration of time.

11. The method of claim 10, wherein the extended period of time is one month to six months.

12. The method of claim 1, wherein the sensing electrodes are disposed on a wrist of the subject.

13. The method of claim 1, wherein the alert is output from at least one of the wearable device and an external device.

14. The method of claim 1, wherein the alert is output when the average edema index exceeds or falls below a preselected value.

15. The method of claim 1, wherein the alert is an electronic report to a caregiver.

16. The method of claim 1, wherein the alert is an audible or visible report.

17. The method of claim 1, wherein the validated sub-set of impedance measurements comprises at least 40% of the plurality of impedance measurements measured during the period of testing.

18. The method of claim 1, wherein the at least two sensing electrodes are at least a centimeter apart on a skin of the subject.

19. The method of claim 1, further comprising:
securing a band including the at least two sensing electrodes to the skin of the subject, thereby disposing the at least two sensing electrodes at different points on the subject.

20. A wearable device for monitoring a level of edema of a subject, comprising:
at least two stimulation electrodes;
at least two sensing electrodes;
a signal generator; and
a controller configured to:
control the signal generator to generate a first signal that causes a current to flow between the stimulation electrodes,
measure an impedance between the sensing electrodes at a selected interval of time during a period of testing, thereby providing a plurality of impedance measurements,
validate each of the plurality of impedance measurements against a model set of impedance measurements,
eliminate an impedance measurement from the plurality of impedance measurements if the impedance measurement fails the validating, thereby providing a validated sub-set of impedance measurements,
convert each of the validated sub-set of impedance measurements to an edema index, thereby providing a plurality of edema indices,
average the plurality of edema indices and generate an average edema index for the period of testing, and
generate an alert depending on the average edema index.
